(19) United States
(12) Patent Application Publication
(10) Pub. No.: US 2022/0354689 A1
(43) Pub. Date: Nov. 10, 2022

(54) BLANKET FOR SOOTHING USERS EXPERIENCING HOT FLASHES AND COLD FLASHES

(71) Applicant: Tami Ditkoff, Syosset, NY (US)

(72) Inventor: Tami Ditkoff, Syosset, NY (US)

(21) Appl. No.: 17/736,403

(22) Filed: May 4, 2022

Related U.S. Application Data
(60) Provisional application No. 63/183,985, filed on May 4, 2021.

(51) Int. Cl. A61F 7/00; A61H 23/00; A61H 23/02; A47G 9/02

(52) U.S. Cl. CPC A61F 7/0097; A61H 23/004; A61H 23/0245; A61F 7/0085; A47G 9/0215; A47G 9/0223; A61F 2007/0093; A61F 2007/006; A61H 2201/0207; A61H 2201/0214; A61F 2007/0064; A61H 2201/025

(57) ABSTRACT

Disclosed herein is a blanket for soothing users experiencing hot flashes and cold flashes, in accordance with some embodiments. Accordingly, the blanket may include at least one first blanket portion and at least one second blanket portion. Further, the at least one first blanket portion may include at least one first element and the at least one second blanket portion may include at least one second element. Further, the at least one first blanket portion configured for soothing at least one first effect of the hot flashes experienced by at least one user based on the at least one first element. Further, the at least one second blanket portion may be configured for soothing at least one second effect of the cold flashes experienced by the at least one user based on the at least one second element.

### FIELD OF THE INVENTION

Generally, the present disclosure relates to the field of apparel. More specifically, the present disclosure relates to a blanket for soothing users experiencing hot flashes and cold flashes.

### BACKGROUND OF THE INVENTION

Menopause is the time in a woman's life when a woman's period stops. Menopause happens because the woman's ovaries stop producing the hormones estrogen and progesterone. One of the symptoms of menopause is a hot flash, a sudden intense feeling of warmth spreading through the chest, neck, and face. Another symptom of menopause is a cold flash, a tingling, shivery, and cold feeling that can suddenly spread through the body. Similar to the hot flash, the cold flash is likely caused by hormone havoc in the head. To soothe the aforementioned symptoms, different types of blankets are used.

Therefore, there is a need for a blanket for soothing users experiencing hot flashes and cold flashes that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a blanket for soothing users experiencing hot flashes and cold flashes, in accordance with some embodiments. Accordingly, the blanket may include at least one first blanket portion and at least one second blanket portion. Further, the at least one first blanket portion may include at least one first element and the at least one second blanket portion may include at least one second element. Further, the at least one first blanket portion configured for soothing at least one first effect of the hot flashes experienced by at least one user based on the at least one first element. Further, the at least one second blanket portion may be configured for soothing at least one second effect of the cold flashes experienced by the at least one user based on the at least one second element.

Further disclosed herein is a blanket for soothing users experiencing hot flashes and cold flashes, in accordance with some embodiments. Accordingly, the blanket may include at least one first blanket portion and at least one second blanket portion. Further, the at least one first blanket portion may include at least one first element and the at least one second blanket portion may include at least one second element. Further, the at least one first blanket portion configured for soothing at least one first effect of the hot flashes experienced by at least one user based on the at least one first element. Further, the at least one second blanket portion may be configured for soothing at least one second effect of the cold flashes experienced by the at least one user based on the at least one second element. Further, the at least one first element may include at least one cotton fabric. Further, the at least one second element may include at least one polyester fabric. Further, the soothing of the at least one first effect of the hot flashes may be further based on the at least one cotton fabric. Further, the soothing of the at least one second effect of the cold flashes may be further based on the at least one polyester fabric.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a front top perspective view of a blanket for soothing users experiencing hot flashes and cold flashes, in accordance with some embodiments.

FIG. 2 is a side top perspective view of the blanket, in accordance with some embodiments.

FIG. 3 is a front top perspective view of the blanket, in accordance with some embodiments.

FIG. 4 is a front top perspective view of the blanket, in accordance with some embodiments.

FIG. 5 is a front top perspective view of the blanket, in accordance with some embodiments.

FIG. 6 is a front top perspective view of the blanket, in accordance with some embodiments.

FIG. 7 is a front top perspective view of the blanket, in accordance with some embodiments.

FIG. 8 is a front top perspective view of the blanket, in accordance with some embodiments.

FIG. 9 is a front top perspective view of the blanket, in accordance with some embodiments.

FIG. 10 is a front top perspective view of the blanket, in accordance with some embodiments.

FIG. 11 is a front top perspective view of the blanket, in accordance with some embodiments.

FIG. 12 is a front top perspective view of the blanket, in accordance with some embodiments.

FIG. 13 is a front top perspective view of a blanket for soothing users experiencing hot flashes and cold flashes, in accordance with some embodiments.

FIG. 14 is a side top perspective view of the blanket, in accordance with some embodiments.

FIG. 15 is a front top perspective view of the blanket, in accordance with some embodiments.

FIG. 16 is an illustration of an online platform consistent with various embodiments of the present disclosure.

FIG. 17 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a blanket for soothing users experiencing hot flashes and cold flashes, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Overview:

The present disclosure describes a blanket for soothing users experiencing hot flashes and cold flashes.

Further, the present disclosure relates generally to a blanket. More specifically, the present disclosure describes a blanket that comprises multiple sides with different fabrics. Further, the blanket may include a Menopause Blanket. Further, the blanket soothes the hot flash and cold flashes symptoms of menopause.

Further, the menopause blanket may include a cotton blanket and a polyester blanket. A user would use the cotton blanket of the menopause blanket during the hot flash, and the polyester blanket of the menopause blanket during the cold flash. Further, the cotton blanket provides a flat and rectangular shape with a dark color, although any desired designs or colors may be used. Further, the cotton blanket is made of, but not limited to, 100% cotton. Similar to the cotton blanket, the polyester blanket provides a flat and rectangular shape but with a lighter color, although any desired designs or colors may be used. Further, the polyester blanket is made of, but not limited to, a polyester blend. Further, the side edges of the cotton blanket and the polyester blanket are sewn.

Further, the present disclosure describes a blanket that comprises multiple sides with different fabrics to soothe the hot flash and cold flash symptoms of menopause. The blanket is also referred to as the “Menopause Blanket,” which takes place of multiple blankets, therefore it is convenient and space efficient.

Further, the menopause blanket is derived from 2 different fabrics. Further, one side of the menopause blanket is 100% cotton and the other side of the menopause blanket is Polyester Blend. Further, the menopause blanket is 2 different fabrics, that are sewn down the middle. Further, the one side is for the users experiencing a hot flash and the other side for the user experiencing freezing right after the hot flash. Further, the menopause blanket provides for a need of the user for multiple blankets of different types. Further, a dimension of the menopause blanket is 80×90 which is evenly split between 2 fabrics or portions of the menopause blanket.

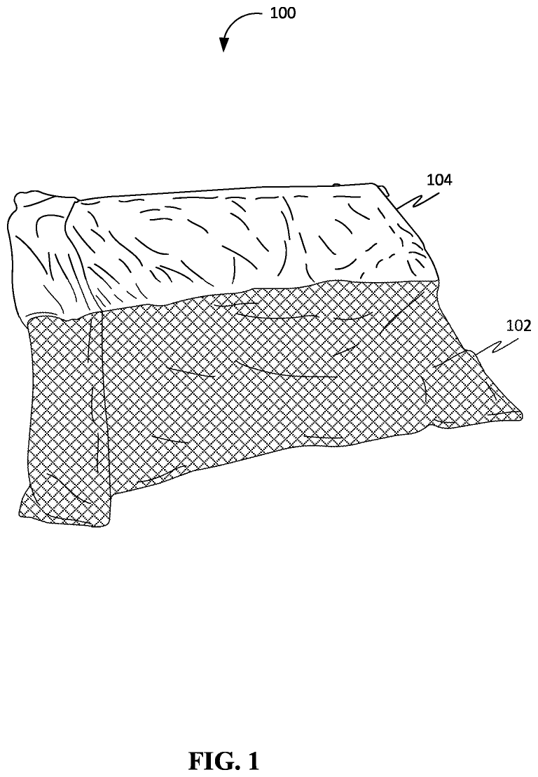

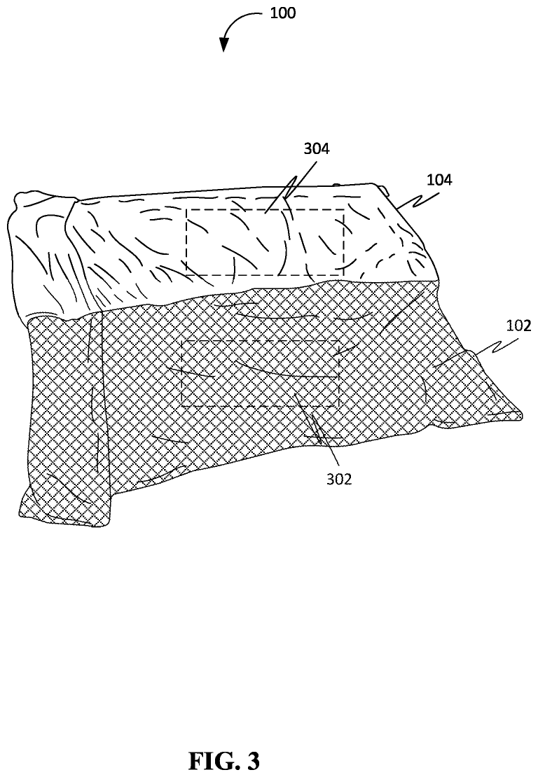

FIG. 1 is a front top perspective view of a blanket 100 for soothing users experiencing hot flashes and cold flashes, in accordance with some embodiments. Further, the blanket 100 may be a menopause blanket. Further, the blanket 100 may include at least one first blanket portion 102 and at least one second blanket portion 104. Further, the at least one first blanket portion 102 may include at least one first element 302, as shown in FIG. 3, and the at least one second blanket portion 104 may include at least one second element 304, as shown in FIG. 3. Further, the at least one first blanket portion 102 configured for soothing at least one first effect of the hot flashes experienced by at least one user based on the at least one first element 302. Further, the at least one first effect of the hot flashes may include a sudden intense feeling of warmth spreading through at least one body part comprising a chest, a neck, a face, etc. Further, the at least one first effect of the hot flashes may include perspiring, sweating, etc. Further, the at least one second blanket portion 104 may be configured for soothing at least one second effect of the cold flashes experienced by the at least one user based on the at least one second element 304. Further, the at least one second effect of the cold flashes may include a sudden intense feeling of cold spreading through the at least one body part of the at least one user. Further, the at least one second effect of the cold flashes may include shivering, tingling, etc.

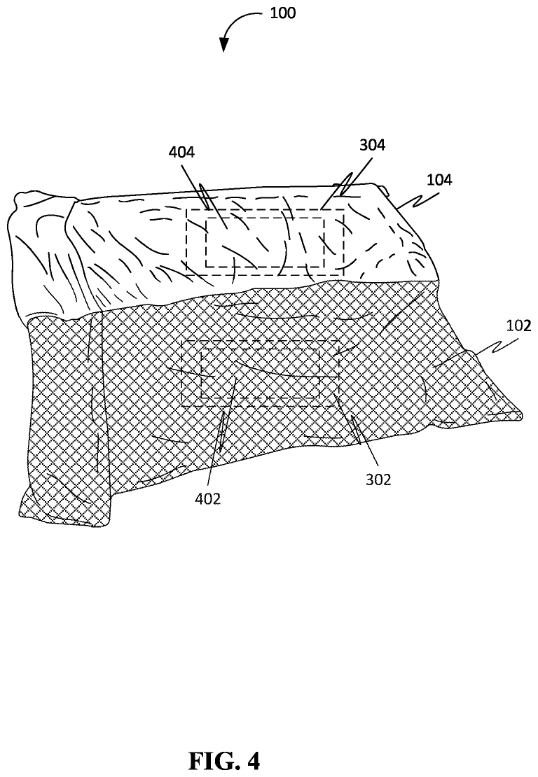

Further, in some embodiments, the at least one first element 302 may include at least one cotton fabric 402, as shown in FIG. 4. Further, the at least one second element 304 may include at least one polyester fabric 404, as shown in FIG. 4. Further, the at least one first blanket portion 102 may be comprised of the at least one cotton fabric 402 and the at least one second blanket portion 104 may be comprised of the at least one polyester fabric 404. Further, the at least one cotton fabric 402 may be 100 percent cotton. Further, the at least one polyester fabric 404 may be blended polyesters. Further, the soothing of the at least one first effect of the hot flashes may be based on the at least one cotton fabric 402. Further, the soothing of the at least one second effect of the cold flashes may be based on the at least one polyester fabric 404. Further, the at least one cotton fabric 402 may absorb sweat secreted by the at least one user during experiencing the sweating of the hot flashes. Further, the at least one cotton fabric 402 may allow airflow through the at least first blanket portion 102 for producing a cooling effect for countering the at least one first effect of the hot flashes. Further, the at least one polyester fabric 404 may restrict the airflow through the at least one second blanket portion 104 for producing a heating effect for countering the at least one second effect of the cold flashes.

Further, in an embodiment, the at least one cotton fabric 402 may be light colored for enhancing the soothing of the at least one first effect of the hot flashes. Further, light coloring of the at least one cotton fabric 402 may allow reflecting heat for countering the at least one first effect of the hot flashes. Further, the at least one polyester fabric 404 may be dark colored for enhancing the soothing of the at least one second effect of the cold flashes. Further, dark coloring of the at least one polyester fabric 404 may allow absorbing the heat for countering the at least one second effect of the cold flashes.

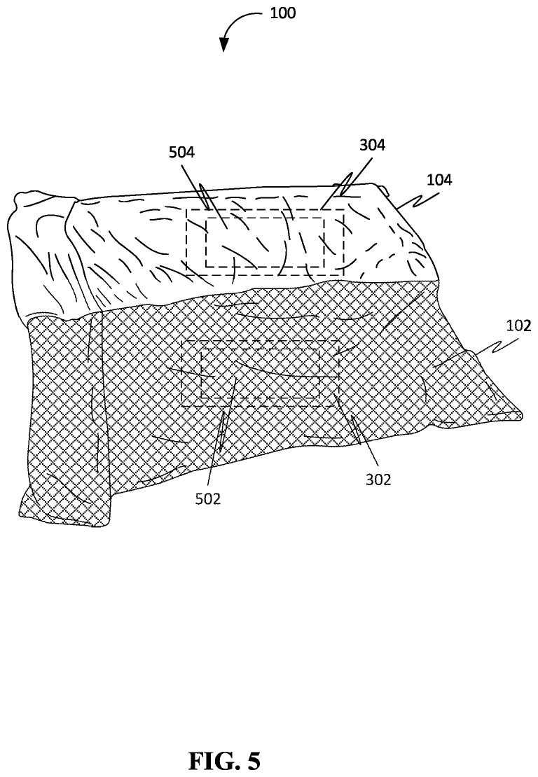

Further, in some embodiments, the at least one first element 302 may include at least one cooling element 502, as shown in FIG. 5. Further, the at least one cooling element 502 may be electrically powered. Further, the at least one cooling element 502 may include a thermoelectric cooler, a cooling device, a cooler, etc. Further, the at least one cooling element 502 may be configured for cooling the at least one first blanket portion 102. Further, the soothing of the at least one first effect of the hot flashes may be based on the cooling of the at least one first blanket portion 102. Further, the at least one second element 304 may include at least one heating element 504, as shown in FIG. 5. Further, the at least one heating element 504 may be configured for heating the at least one second blanket portion 104. Further, the at least one heating element 504 may be electrically powered. Further, the at least one heating element 504 may include a thermoelectric heater, a heating device, a heater, etc. Further, the soothing of the at least one second effect of the cold flashes may be based on the heating of the at least one second blanket portion 104.

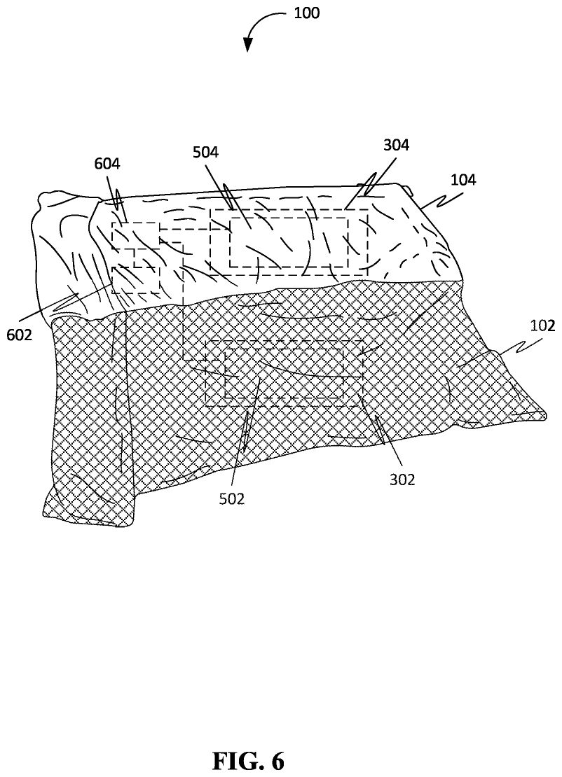

In further embodiments, the blanket 100 may include at least one sensor 602 and a processing device 604, as shown in FIG. 6. Further, the at least one sensor 602 may be disposed in the blanket 100. Further, the at least one sensor 602 may be configured for generating at least one sensor data based on detecting at least one of a physiological parameter and a physical parameter of the at least one user. Further, the physiological parameter may include blood flow, heart rate, blood pressure, hemoglobin levels, cardiac output, arterial oxygen concentration, etc. Further, the physical parameter may include parameters related to activity, posture, and sleep of the at least one user, a body temperature, a bodyweight, etc. Further, the processing device 604 may be communicatively coupled with the at least one sensor 602. Further, the processing device 604 may be configured for analyzing the at least one sensor data. Further, the processing device 604 may be configured for determining a state of the at least one user based on the analyzing. Further, the state may include a first state and a second state. Further, the user experiences the at least one first effect of the hot flashes in the first state and the user experiences the at least one second effect of the cold flashes in the second state. Further, the processing device 604 may be configured for generating at least one command for at least one of the at least one cooling element 502 and the at least one heating element 504 based on the state. Further, at least one of the at least one cooling element 502 and the at least one heating element 504 may be communicatively coupled with the processing device 604. Further, at least one of the cooling of the at least one first blanket portion 102 and the heating of the at least one second blanket portion 104 may be based on the at least one command.

Further, in an embodiment, the blanket 100 may be disposed over the at least one user. Further, the analyzing of the at least one sensor data may include analyzing the at least one sensor data using at least one machine learning model. Further, the processing device 604 may be configured for identifying at least one body part of the at least one user experiencing at least one of the at least one first effect of the hot flashes and the at least one second effect of the cold flashes based on the analyzing of the at least one sensor data using the at least one machine learning model. Further, the processing device 604 may be configured for identifying at least a part of at least one of the at least one first blanket portion 102 and the at least one second blanket portion 104 corresponding to the at least one body part based on the identifying of the at least one body part. Further, the generating of the at least one command for at least one of the at least one cooling element 502 and the at least one heating element 504 may be based on the identifying of at least the part of at least one of the at least one first blanket portion 102 and the at least one second blanket portion 104. Further, at least one of the cooling of the at least one first blanket portion 102 and the heating of the at least one second blanket portion 104 may include at least one of the cooling of at least the part of the at least one first blanket portion 102 and the heating of at least the part of the at least one second blanket portion 104 based on the at least one command.

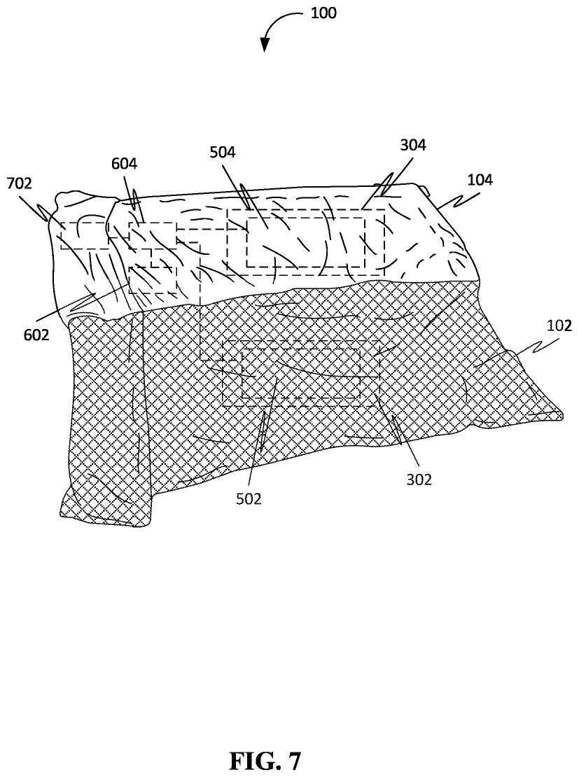

In further embodiments, the blanket 100 may include at least one biological sensor 702, as shown in FIG. 7. Further, the at least one biological sensor 702 may be disposed in the blanket 100. Further, the at least one biological sensor 702 may be communicatively coupled with the processing device 604. Further, the at least one biological sensor 702 may be configured for generating at least one biological sensor data based on detecting at least one biological metric of the at least one user. Further, the at least one biological metric may include a level of at least one human body hormone. Further, the at least one body hormone may include estrogen, progesterone, estradiol, thyroid hormone, cortisol, etc. Further, the processing device 604 may be configured for analyzing the at least one biological sensor data. Further, the determining of the state of the at least one user may be based on the analyzing of the at least one biological sensor data. Further, the at least one biological sensor may include a biosensor.

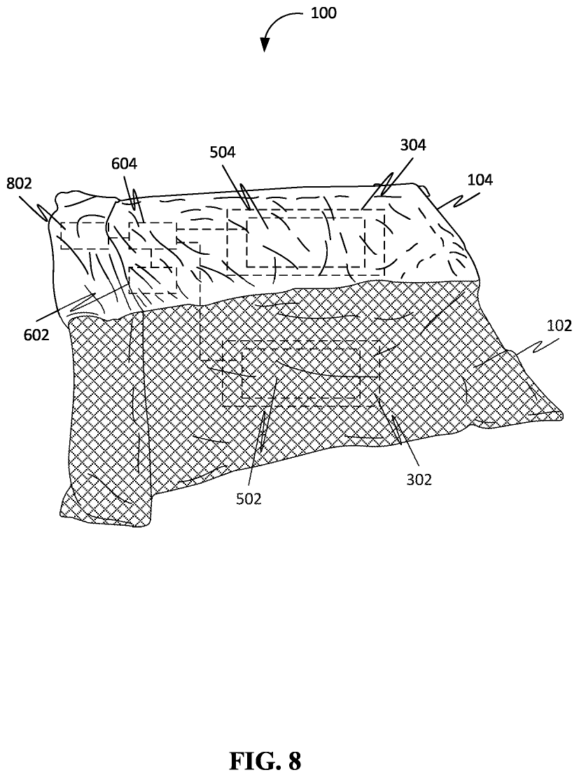

Further, in an embodiment, the analyzing of the at least one sensor data may include analyzing the at least one sensor data using at least one first machine learning model. Further, the processing device 604 may be configured for predicting a future state of the at least one user based on the analyzing of the at least one sensor data using the at least one first machine learning model. Further, the future state corresponds to the state. Further, the processing device 604 may be configured for generating at least one alert for the future state based on the predicting. Further, the processing device 604 may be communicatively coupled with at least one output device 802, as shown in FIG. 8. Further, the at least one output device 802 may be configured for presenting the at least one alert.

Further, in some embodiments, the at least one first blanket portion 102 may include a first half of the blanket 100 and the at least one second blanket portion 104 may include a second half of the blanket 100. Further, the first half and the second half form the blanket 100.

Further, in an embodiment, the at least one first blanket portion 102 may be fixedly attached to the at least one second blanket portion 104. Further, the at least one first blanket portion 102 may be sewed to the at least one second blanket portion 104 for fixedly attaching the at least one first blanket portion 102 to the at least one second blanket portion 104.

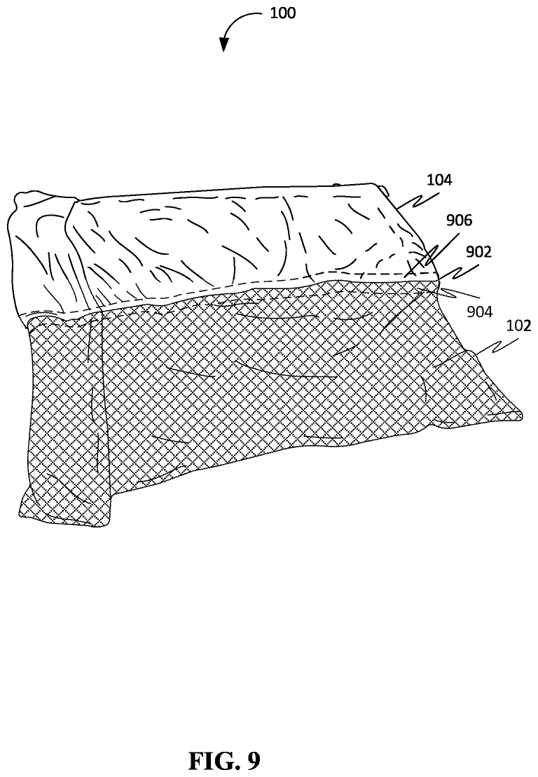

Further, in an embodiment, the at least one first blanket portion 102 may be detachably attached to the at least one second blanket portion 104 using at least one attachment element 902, as shown in FIG. 9. Further, the at least one attachment element 902 may include a first attachment element 904, as shown in FIG. 9, comprised in the at least one first blanket portion 102 and a second attachment element 906, as shown in FIG. 9, comprised in the at least one second blanket portion 104. Further, the at least one attachment element 902 may include a zip, a hook and loop, a fastener. Further, the first attachment element 904 may include a hook and the second attachment element 906 may include a loop. Further, the first attachment element 904 disengageably engages with the second attachment element 906 for detachably attaching the at least one first blanket portion 102 to the at least one second blanket portion 104.

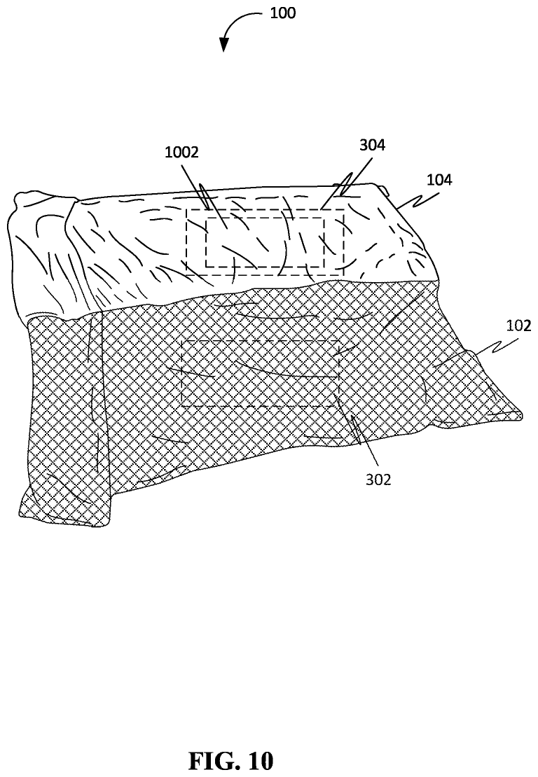

Further, in some embodiments, the at least one first element 302 may include at least one airflow device 1002, as shown in FIG. 10. Further, the at least one airflow device may be disposed in the blanket 100. Further, the at least one airflow device 1002 may be configured for discharging at least one airstream through the at least one first blanket portion 102. Further, the soothing of the at least one first effect of the hot flashes may be based on the discharging. Further, the at least one airflow device 1002 may include a fan assembly comprising a fan and a tubing comprising at least one inlet and at least one outlet. Further, the fan intakes the air from the at least one inlet and expels the air from the at least one outlet for the discharging of the at least one airstream.

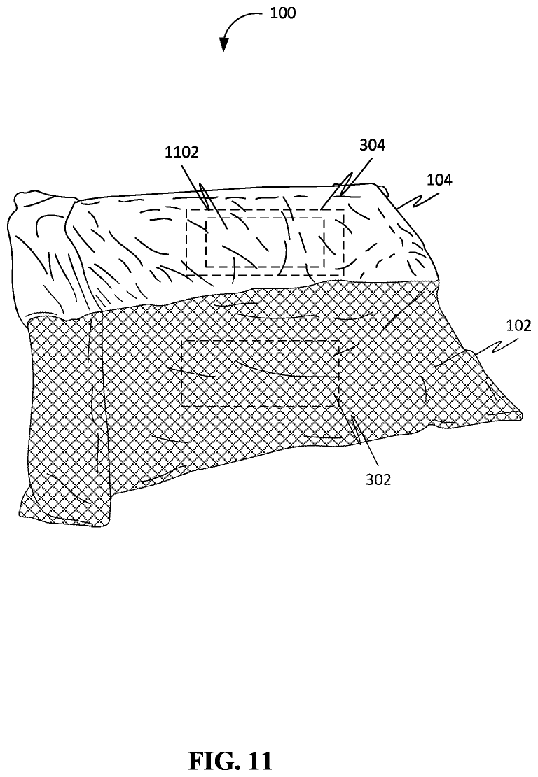

Further, in some embodiments, the at least one first element 302 may include at least one absorbing element 1102, as shown in FIG. 11. Further, the at least one absorbing element 1102 may be configured for absorbing at least one liquid without getting wet. Further, the soothing of the at least one first effect of the hot flashes may be based on the absorbing. Further, the at least one absorbing element 1102 may include an absorbent, a silica gel, hydrogel, etc.

Further, in some embodiments, the at least one second element 304 may include at least one vibrating device 1202. Further, the at least one vibrating device 1202 may be configured for vibrating the at least one second blanket portion 104. Further, the soothing of the at least one second effect of the cold flashes may be based on the vibrating. Further, the at least one vibrating device 1202 may be electrically powered. Further, the at least one vibrating device 1202 may include a mechanical vibrator, a piezoelectric vibrator, etc.

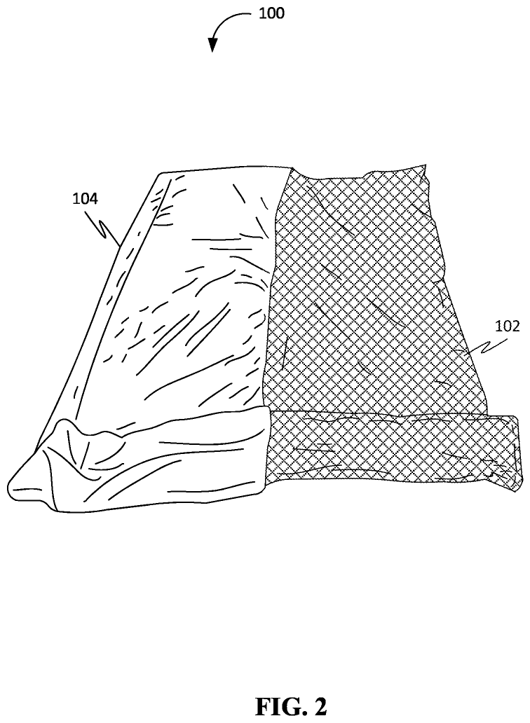

FIG. 2 is a side top perspective view of the blanket 100, in accordance with some embodiments.

FIG. 3 is a front top perspective view of the blanket 100, in accordance with some embodiments.

FIG. 4 is a front top perspective view of the blanket 100, in accordance with some embodiments.

FIG. 5 is a front top perspective view of the blanket 100, in accordance with some embodiments.

FIG. 6 is a front top perspective view of the blanket 100, in accordance with some embodiments.

FIG. 7 is a front top perspective view of the blanket 100, in accordance with some embodiments.

FIG. 8 is a front top perspective view of the blanket 100, in accordance with some embodiments.

FIG. 9 is a front top perspective view of the blanket 100, in accordance with some embodiments.

FIG. 10 is a front top perspective view of the blanket 100, in accordance with some embodiments.

FIG. 11 is a front top perspective view of the blanket 100, in accordance with some embodiments.

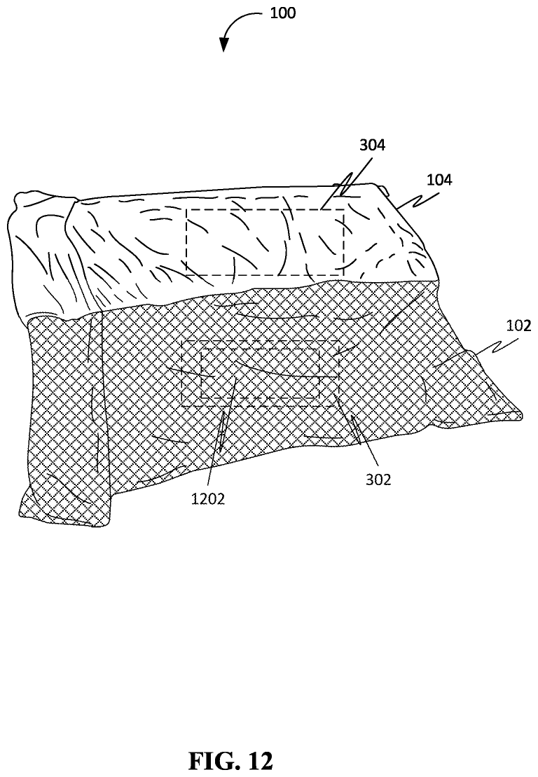

FIG. 12 is a front top perspective view of the blanket 100, in accordance with some embodiments.

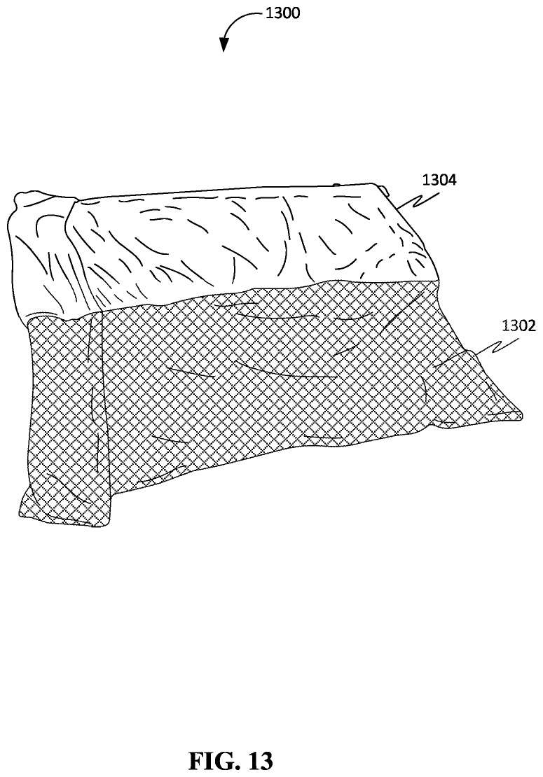

FIG. 13 is a front top perspective view of a blanket 1300 for soothing users experiencing hot flashes and cold flashes, in accordance with some embodiments. Further, the blanket 1300 may include at least one first blanket portion 1302 and at least one second blanket portion 1304. Further, the at least one first blanket portion 1302 may include at least one first element 1502 and the at least one second blanket portion 1304 may include at least one second element 1504. Further, the at least one first blanket portion 1302 configured for soothing at least one first effect of the hot flashes experienced by at least one user based on the at least one first element 1502. Further, the at least one second blanket portion 1304 may be configured for soothing at least one second effect of the cold flashes experienced by the at least one user based on the at least one second element 1504. Further, the at least one first element 1502 may include at least one cotton fabric 1506. Further, the at least one second element 1504 may include at least one polyester fabric 1508. Further, the soothing of the at least one first effect of the hot flashes may be based on the at least one cotton fabric 1506. Further, the soothing of the at least one second effect of the cold flashes may be based on the at least one polyester fabric 1508.

Further, in some embodiments, the at least one cotton fabric 1506 may be light colored for enhancing the soothing of the at least one first effect of the hot flashes. Further, the at least one polyester fabric 1508 may be dark colored for enhancing the soothing of the at least one second effect of the cold flashes.

Further, in some embodiments, the at least one first element 1502 may include at least one cooling element. Further, the at least one cooling element may be configured for cooling the at least one first blanket portion 1302. Further, the soothing of the at least one first effect of the hot flashes may be based on the cooling of the at least one first blanket portion 1302. Further, the at least one second element 1504 may include at least one heating element. Further, the at least one heating element may be configured for heating the at least one second blanket portion 1304. Further, the soothing of the at least one second effect of the cold flashes may be based on the heating of the at least one second blanket portion 1304.

Further, in some embodiments, the at least one first blanket portion 1302 may include a first half of the blanket 1300 and the at least one second blanket portion 1304 may include a second half of the blanket 1300. Further, the first half and the second half form the blanket 1300.

Further, in an embodiment, the at least one first blanket portion 1302 may be fixedly attached to the at least one second blanket portion 1304.

Further, in an embodiment, the at least one first blanket portion 1302 may be detachably attached to the at least one second blanket portion 1304 using at least one attachment element. Further, the at least one attachment element may include a first attachment element comprised in the at least one first blanket portion 1302 and a second attachment element comprised in the at least one second blanket portion 1304. Further, the first attachment element disengageably engages with the second attachment element for detachably attaching the at least one first blanket portion 1302 to the at least one second blanket portion 1304.

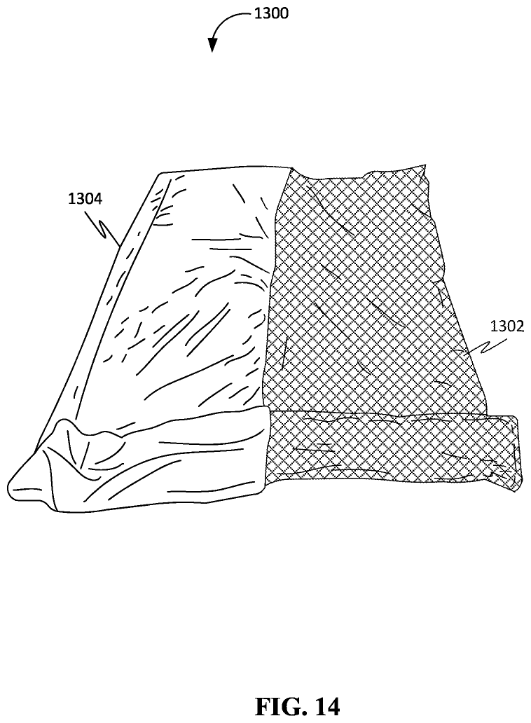

FIG. 14 is a side top perspective view of the blanket 1300, in accordance with some embodiments.

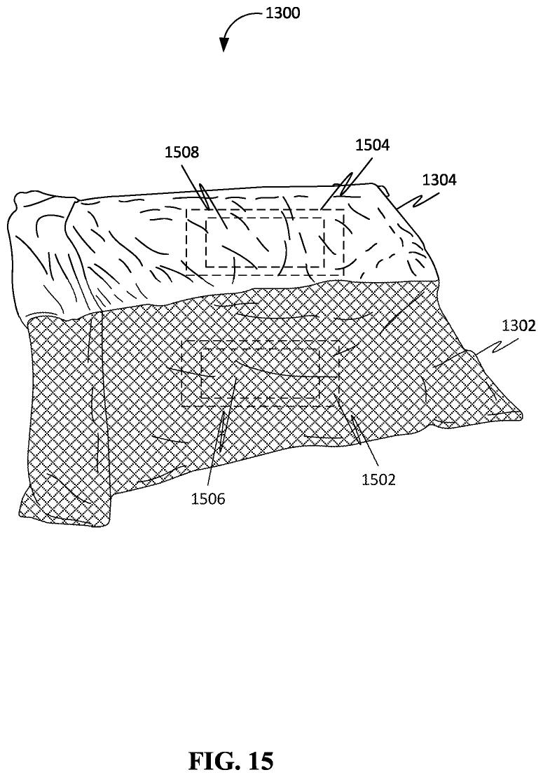

FIG. 15 is a front top perspective view of the blanket 1300, in accordance with some embodiments.

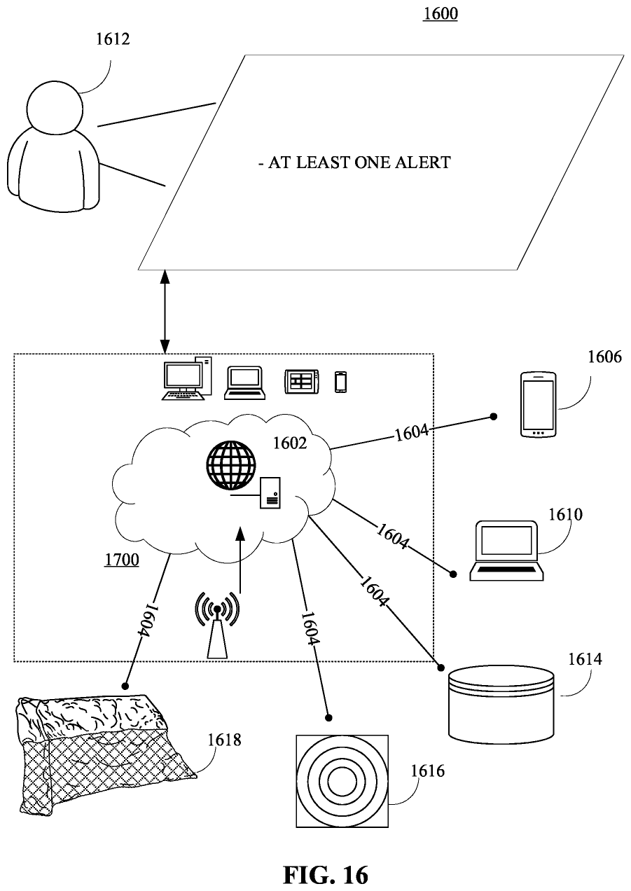

FIG. 16 is an illustration of an online platform 1600 consistent with various embodiments of the present disclosure. By way of non-limiting example, the online platform 1600 to facilitate managing a blanket for soothing users experiencing hot flashes and cold flashes may be hosted on a centralized server 1602, such as, for example, a cloud computing service. The centralized server 1602 may communicate with other network entities, such as, for example, a mobile device 1606 (such as a smartphone, a laptop, a tablet computer, etc.), other electronic devices 1610 (such as desktop computers, server computers, etc.), databases 1614, sensors 1616, and a blanket 1618 (such as the blanket 100 and the blanket 1300) over a communication network 1604, such as, but not limited to, the Internet. Further, users of the online platform 1600 may include relevant parties such as, but not limited to, end-users, administrators, service providers, service consumers, and so on. Accordingly, in some instances, electronic devices operated by the one or more relevant parties may be in communication with the platform.

A user 1612, such as the one or more relevant parties, may access online platform 1600 through a web based software application or browser. The web based software application may be embodied as, for example, but not be limited to, a website, a web application, a desktop application, and a mobile application compatible with a computing device 1700.

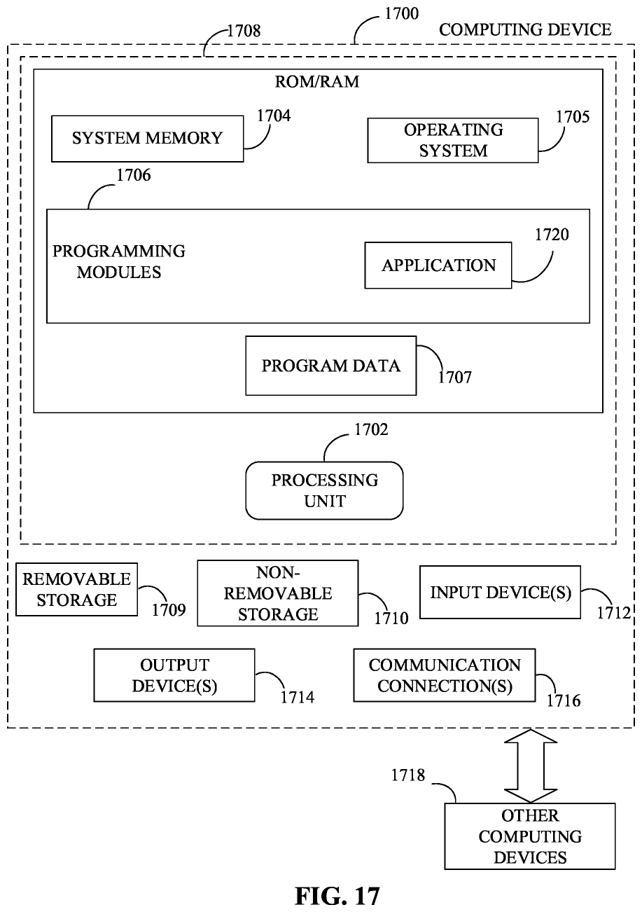

With reference to FIG. 17, a system consistent with an embodiment of the disclosure may include a computing device or cloud service, such as computing device 1700. In a basic configuration, computing device 1700 may include at least one processing unit 1702 and a system memory 1704. Depending on the configuration and type of computing device, system memory 1704 may comprise, but is not limited to, volatile (e.g. random-access memory (RAM)), non-volatile (e.g. read-only memory (ROM)), flash memory, or any combination. System memory 1704 may include operating system 1705, one or more programming modules 1706, and may include a program data 1707. Operating system 1705, for example, may be suitable for controlling computing device 1700's operation. In one embodiment, programming modules 1706 may include image-processing module, machine learning module. Furthermore, embodiments of the disclosure may be practiced in conjunction with a graphics library, other operating systems, or any other application program and is not limited to any particular application or system. This basic configuration is illustrated in FIG. 17 by those components within a dashed line 1708.

Computing device 1700 may have additional features or functionality. For example, computing device 1700 may also include additional data storage devices (removable and/or non-removable) such as, for example, magnetic disks, optical disks, or tape. Such additional storage is illustrated in FIG. 17 by a removable storage 1709 and a non-removable storage 1710. Computer storage media may include volatile and non-volatile, removable and non-removable media implemented in any method or technology for storage of information, such as computer-readable instructions, data structures, program modules, or other data. System memory 1704, removable storage 1709, and non-removable storage 1710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1700. Any such computer storage media may be part of device 1700. Computing device 1700 may also have input device(s) 1712 such as a keyboard, a mouse, a pen, a sound input device, a touch input device, a location sensor, a camera, a biometric sensor, etc. Output device(s) 1714 such as a display, speakers, a printer, etc. may also be included. The aforementioned devices are examples and others may be used.

Computing device 1700 may also contain a communication connection 1716 that may allow device 1700 to communicate with other computing devices 1718, such as over a network in a distributed computing environment, for example, an intranet or the Internet. Communication connection 1716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1704, including operating system 1705. While executing on processing unit 1702, programming modules 1706 (e.g., application 1720 such as a media player) may perform processes including, for example, one or more stages of methods, algorithms, systems, applications, servers, databases as described above. The aforementioned process is an example, and processing unit 1702 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A blanket for soothing users experiencing hot flashes and cold flashes, the blanket comprising:
at least one first blanket portion; and
at least one second blanket portion, wherein the at least one first blanket portion comprises at least one first element and the at least one second blanket portion comprises at least one second element, wherein the at least one first blanket portion configured for soothing at least one first effect of the hot flashes experienced by at least one user based on the at least one first element, wherein the at least one second blanket portion is configured for soothing at least one second effect of the cold flashes experienced by the at least one user based on the at least one second element.

2. The blanket of claim 1, wherein the at least one first element comprises at least one cotton fabric, wherein the at least one second element comprises at least one polyester fabric, wherein the soothing of the at least one first effect of the hot flashes is further based on the at least one cotton fabric, wherein the soothing of the at least one second effect of the cold flashes is further based on the at least one polyester fabric.

3. The blanket of claim 2, wherein the at least one cotton fabric is light colored for enhancing the soothing of the at least one first effect of the hot flashes, wherein the at least one polyester fabric is dark colored for enhancing the soothing of the at least one second effect of the cold flashes.

4. The blanket of claim 1, wherein the at least one first element further comprises at least one cooling element, wherein the at least one cooling element is configured for cooling the at least one first blanket portion, wherein the soothing of the at least one first effect of the hot flashes is based on the cooling of the at least one first blanket portion, wherein the at least one second element further comprises at least one heating element, wherein the at least one heating element is configured for heating the at least one second blanket portion, wherein the soothing of the at least one second effect of the cold flashes is further based on the heating of the at least one second blanket portion.

5. The blanket of claim 4 further comprising:
at least one sensor disposed in the blanket, wherein the at least one sensor is configured for generating at least one sensor data based on detecting at least one of a physiological parameter and a physical parameter of the at least one user; and
a processing device communicatively coupled with the at least one sensor, wherein the processing device is configured for:
analyzing the at least one sensor data;
determining a state of the at least one user based on the analyzing, wherein the state comprises a first state and a second state, wherein the user experiences the at least one first effect of the hot flashes in the first state and the user experiences the at least one second effect of the cold flashes in the second state; and
generating at least one command for at least one of the at least one cooling element and the at least one heating element based on the state, wherein at least one of the at least one cooling element and the at least one heating element is communicatively coupled with the processing device, wherein at least one of the cooling of the at least one first blanket portion and the heating of the at least one second blanket portion is further based on the at least one command.

6. The blanket of claim 5, wherein the blanket is disposed over the at least one user, wherein the analyzing of the at least one sensor data comprises analyzing the at least one sensor data using at least one machine learning model, wherein the processing device is further configured for:
identifying at least one body part of the at least one user experiencing at least one of the at least one first effect of the hot flashes and the at least one second effect of the cold flashes based on the analyzing of the at least one sensor data using the at least one machine learning model; and
identifying at least a part of at least one of the at least one first blanket portion and the at least one second blanket portion corresponding to the at least one body part based on the identifying of the at least one body part, wherein the generating of the at least one command for at least one of the at least one cooling element and the at least one heating element is further based on the identifying of at least the part of at least one of the at least one first blanket portion and the at least one second blanket portion, wherein at least one of the cooling of the at least one first blanket portion and the heating of the at least one second blanket portion comprises at least one of the cooling of at least the part of the at least one first blanket portion and the heating of at least the part of the at least one second blanket portion based on the at least one command.

7. The blanket of claim 5 further comprising at least one biological sensor disposed in the blanket, wherein the at least one biological sensor is communicatively coupled with the processing device, wherein the at least one biological sensor is configured for generating at least one biological sensor data based on detecting at least one biological metric of the at least one user, wherein the processing device is further configured for analyzing the at least one biological sensor data, wherein the determining of the state of the at least one user is further based on the analyzing of the at least one biological sensor data.

8. The blanket of claim 5, wherein the analyzing of the at least one sensor data comprises analyzing the at least one sensor data using at least one first machine learning model, wherein the processing device is further configured for:
predicting a future state of the at least one user based on the analyzing of the at least one sensor data using the at least one first machine learning model; and
generating at least one alert for the future state based on the predicting, wherein the processing device is communicatively coupled with at least one output device, wherein the at least one output device is configured for presenting the at least one alert.

9. The blanket of claim 1, wherein the at least one first blanket portion comprises a first half of the blanket and the at least one second blanket portion comprises a second half of the blanket, wherein the first half and the second half form the blanket.

10. The blanket of claim 9, wherein the at least one first blanket portion is fixedly attached to the at least one second blanket portion.

11. The blanket of claim 9, wherein the at least one first blanket portion is detachably attached to the at least one second blanket portion using at least one attachment element, wherein the at least one attachment element comprises a first attachment element comprised in the at least one first blanket portion and a second attachment element comprised in the at least one second blanket portion, wherein the first attachment element disengageably engages with the second attachment element for detachably attaching the at least one first blanket portion to the at least one second blanket portion.

12. The blanket of claim 1, wherein the at least one first element further comprises at least one airflow device, wherein the at least one airflow device is configured for discharging at least one airstream through the at least one first blanket portion, wherein the soothing of the at least one first effect of the hot flashes is further based on the discharging.

13. The blanket of claim 1, wherein the at least one first element further comprises at least one absorbing element, wherein the at least one absorbing element is configured for absorbing at least one liquid without getting wet, wherein the soothing of the at least one first effect of the hot flashes is further based on the absorbing.

14. The blanket of claim 1, wherein the at least one second element further comprises at least one vibrating device, wherein the at least one vibrating device is configured for vibrating the at least one second blanket portion, wherein the soothing of the at least one second effect of the cold flashes is further based on the vibrating.

15. A blanket for soothing users experiencing hot flashes and cold flashes, the blanket comprising:
at least one first blanket portion; and
at least one second blanket portion, wherein the at least one first blanket portion comprises at least one first element and the at least one second blanket portion comprises at least one second element, wherein the at least one first blanket portion configured for soothing at least one first effect of the hot flashes experienced by at least one user based on the at least one first element, wherein the at least one second blanket portion is configured for soothing at least one second effect of the cold flashes experienced by the at least one user based on the at least one second element, wherein the at least one first element comprises at least one cotton fabric, wherein the at least one second element comprises at least one polyester fabric, wherein the soothing of the at least one first effect of the hot flashes is further based on the at least one cotton fabric, wherein the soothing of the at least one second effect of the cold flashes is further based on the at least one polyester fabric.

16. The blanket of claim 15, wherein the at least one cotton fabric is light colored for enhancing the soothing of the at least one first effect of the hot flashes, wherein the at least one polyester fabric is dark colored for enhancing the soothing of the at least one second effect of the cold flashes.

17. The blanket of claim 15, wherein the at least one first element further comprises at least one cooling element, wherein the at least one cooling element is configured for cooling the at least one first blanket portion, wherein the soothing of the at least one first effect of the hot flashes is based on the cooling of the at least one first blanket portion, wherein the at least one second element further comprises at least one heating element, wherein the at least one heating element is configured for heating the at least one second blanket portion, wherein the soothing of the at least one second effect of the cold flashes is further based on the heating of the at least one second blanket portion.

18. The blanket of claim 15, wherein the at least one first blanket portion comprises a first half of the blanket and the at least one second blanket portion comprises a second half of the blanket, wherein the first half and the second half form the blanket.

19. The blanket of claim 18, wherein the at least one first blanket portion is fixedly attached to the at least one second blanket portion.

20. The blanket of claim 18, wherein the at least one first blanket portion is detachably attached to the at least one second blanket portion using at least one attachment element, wherein the at least one attachment element comprises a first attachment element comprised in the at least one first blanket portion and a second attachment element comprised in the at least one second blanket portion, wherein the first attachment element disengageably engages with the second attachment element for detachably attaching the at least one first blanket portion to the at least one second blanket portion.
